(19) United States
(12) Patent Application Publication
(10) Pub. No.: US 2025/0120970 A1
(43) Pub. Date: Apr. 17, 2025

(54) PHARMACEUTICAL COMPOSITION INCLUDING PHTHALAZINONE DERIVATIVE FOR CO-ADMINISTRATION WITH ANTICANCER DRUG

(71) Applicant: IDIENCE CO., LTD., Seoul (KR)

(72) Inventors: Won Sik LEE, Seoul (KR); Kyoung Soo HA, Seoul (KR); Eun-Jihn ROH, Seoul (KR); Myongjae LEE, Seoul (KR); Minju HONG, Seoul (KR); Sungyeon CHOI, Seoul (KR); Jeong Eun KIM, Seoul (KR); In-Gyu JE, Hwaseong-si, Gyeonggi-do (KR); Yeongran YOO, Hwaseong-si, Gyeonggi-do (KR); Jong-Ha LIM, Hwaseong-si, Gyeonggi-do (KR)

(73) Assignee: IDIENCE CO., LTD., Seoul (KR)

(21) Appl. No.: 18/730,930

(22) PCT Filed: Jan. 25, 2023
(86) PCT No.: PCT/IB2023/000030

§ 371 Date: Jul. 22, 2024

Related U.S. Application Data
(60) Provisional application No. 63/302,834, filed on Jan. 25, 2022.

(51) Int. Cl. A61K 31/502; A61K 31/4745; A61K 31/555; A61P 35/00

(52) U.S. Cl. CPC A61K 31/502; A61K 31/4745; A61K 31/555; A61P 35/00

(57) ABSTRACT

A combination of pharmaceutical compositions for co-administration is disclosed. The combination includes (i) a first pharmaceutical composition containing a phthalazinone derivative and (ii) a second pharmaceutical composition containing at least one anticancer drug. The combination exhibits an excellent synergistic effect compared to the conventional administration of an anticancer drug alone. The first and the second pharmaceutical compositions can be in a form of a mixture, or are each formulated and administered simultaneously or sequentially.

### CROSS-REFERENCE TO RELATED APPLICATIONS

This application is a National of International Application No. PCT/IB2023/000030 filed Jan. 25, 2023, claiming priority based on U.S. Provisional Patent Application No. 63/302,834, filed on Jan. 25, 2022, the entire content of which is incorporated by reference herein.

### TECHNICAL FIELD

The present disclosure relates to a pharmaceutical composition for co-administration including a phthalazinone derivative used for prevention or treatment of cancer. More particularly, the present disclosure relates to a pharmaceutical composition for co-administration including a phthalazinone derivative and an anticancer drug.

The present disclosure also relates to a pharmaceutical composition for co-administration including a phthalazinone derivative used for prevention or treatment of cancer, and irinotecan and/or oxaliplatin.

The present disclosure also relates to a method of treating cancer in a patient, the method including administering a phthalazinone derivative used for prevention or treatment of cancer in combination with irinotecan and/or oxaliplatin.

### BACKGROUND

Although conventional therapies (such as, radiation therapy, chemotherapy, targeted therapy, immunotherapy, and hormone therapy) provide some cancer patients a complete response (complete remission), most cancer patients have a moderate response or are non-responsive, and a certain number of cancer patients still develop disease progression. Thus, attempts to treat some patients with advanced cancer using such conventional therapies or maintenance therapies are limited.

Recently, a treatment method including administering a PARP inhibitor (PARP-1, PARP-2, and PARP-3; collectively referred to as “PARP”), which inhibits poly(ADP-ribose) polymerase, has been used to successfully address the emergence of cancer cells having heterogeneity and treatment-resistance within a cancer cell population.

PARP belongs to a family of enzymes involved in DNA repair and consists of 18 components. Enzymes belonging thereto are located in the cell nucleus, and are key enzymes in the DNA repair mechanism of human cells when damage occurs to single-stranded DNA of cancer cell.

Such PARP inhibitors induce and increase DNA damage in cells through a homologous recombination repair process. In cancer cells that already have homologous recombination deficiency (HRD), PARP inhibitors block the action of PARP in order to accumulate DNA damage in cancer cells and induce cell death.

Examples of PARP inhibitors are Olaparib (AZD-2281), Rucaparib (AG-014699), Niraparib (MK-4827), Talazoparib (BMN-673), Veliparib (ATB-888), Fluzoparib (SHR-3162), Pamiparib (BGN-290), Senaparib (IMP-4297), Stenoparib (2X-121), AZD-5305, and the like. In particular, Olaparib is a strong inhibitor of human PARP, and inhibits in vitro proliferation of specific tumor cell lines and in vivo proliferation of tumors using a monotherapy or a combination therapy with established chemotherapy (Cancers 2020, 12 (2), 334; doi: 10.3390/cancers12020334).

However, when Olaparib is used as a therapeutic agent, cancer cells that have acquired resistance to Olaparib in vivo appear, thereby reducing therapeutic effect. Accordingly, the development of new therapeutic agents that can effectively treat cancer having resistance to certain PARP inhibitors, such as Olaparib, is urgently required.

A monotherapy using a particular PARP inhibitor alone has shown excellent efficacy and safety profile in clinical practice, but major limitations thereof are the need for HRD and rapid emergence of resistance. Many tumors that initially respond to a therapy using PARP inhibitors eventually relapse by restoring homologous recombination activity or through compensatory mutations that stimulate the activity of alternative repair pathways. Therefore, use of a particular PARP inhibitor is limited to specific tumor types, and cannot be used in any cancer treatment.

U.S. Pat. No. 9,682,973, which is incorporated herein by reference in its entirety, discloses a PARP inhibitor compound represented by Formula 1, which is a phthalazinone derivative having anticancer activity, or a pharmaceutically acceptable salt thereof. In particular, the hydrochloride salt form of the compound represented by Formula 1 is one of the promising therapeutic drug candidates as anticancer drugs.

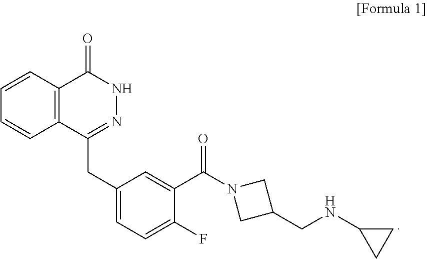 [SMILES: O=C(c1cc(Cc2n[nH]c(=O)c3ccccc23)ccc1F)N1CC(CNC2CC2)C1]

### DESCRIPTION OF DISCLOSURE

### Technical Problem

An object of the present disclosure is to provide a pharmaceutical composition for co-administration, including a phthalazinone derivative as a poly(ADP-ribose) polymerase (PARP) inhibitor having excellent activity, and at least one anticancer drug.

An object of the present disclosure is to provide a pharmaceutical composition for co-administration, including a compound represented by Formula 1, which is a phthalazinone derivative, or a pharmaceutically acceptable salt thereof.

An object of the present disclosure is to provide a kit including, as a concomitant agent for simultaneous, separate, or sequential use in cancer treatment, a phthalazinone derivative as a PARP inhibitor having excellent activity, and at least one anticancer drug capable of co-administration.

An object of the present disclosure is to provide a method of treating cancer, the method including co-administering a PARP inhibitor and at least one anticancer drug simultaneously or sequentially for the treatment of cancer. The method is not limited to those related to homologous recombination deficiency (HRD).

In addition, the present disclosure provides a synergistic therapeutic effect on tumors by providing co-administration of the compound represented by Formula 1 or the pharmaceutically acceptable salt thereof and an anticancer drug.

### Solution to Problem

An aspect of the present disclosure provides a pharmaceutical composition for co-administration, including, as active ingredients, (i) a compound represented by Formula 1 {4-[3-(3-[(cyclopropylamino)methyl]azetidine-1-carbonyl)-4-fluorobenzyl]phthalazine-1 (2H)-on} or a pharmaceutically acceptable salt thereof, and (ii) at least one anticancer drug:

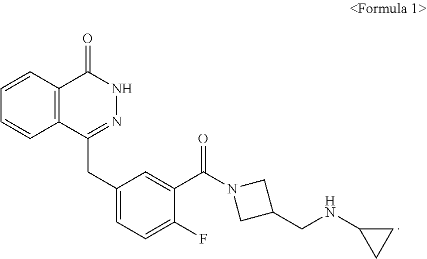 [SMILES: O=C(c1cc(Cc2n[nH]c(=O)c3ccccc23)ccc1F)N1CC(CNC2CC2)C1]

In another aspect of the present disclosure provides a combination of pharmaceutical compositions for co-administration to prevent or treat cancer, wherein the combination comprises: (i) a first pharmaceutical composition comprising, as an active ingredient, a compound represented by Formula 1 or a pharmaceutically acceptable salt thereof;

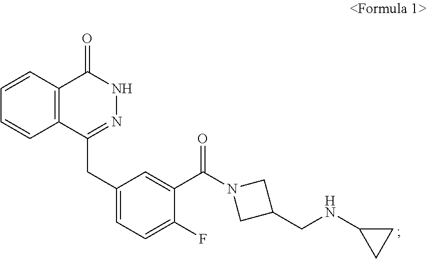 [SMILES: O=C(c1cc(Cc2n[nH]c(=O)c3ccccc23)ccc1F)N1CC(CNC2CC2)C1]

and
(ii) a second pharmaceutical composition comprising, as an active ingredient, at least one anticancer drug.

In the present disclosure, the compound represented by Formula 1 may be a type of phthalazinone derivative.

In the present disclosure, the term “compound represented by Formula 1” may be also simply referred to as “compound of Formula 1” or “compound of the present disclosure”. Similarly, the term “salt of the compound represented by Formula 1” may be also referred to as “salt of the compound of Formula 1” or “salt compound of Formula 1”. For example, when the compound represented by Formula 1 is present in the form of hydrochloride, the compound may be referred to as “hydrochloride of the compound of Formula 1” or “hydrochloride compound of Formula 1”. Such a salt form may include inorganic acid (hydrochloric acid, hydrobromic acid, phosphoric acid, nitric acid, sulfuric acid, etc.), organic carboxylic acid (acetic acid, haloacetic acid such as trifluoroacetic acid, propionic acid, maleic acid, succinic acid, malic acid, citric acid, tartaric acid, salicylic acid, etc.), sugar acid (glucuronic acid, galacturonic acid, gluconic acid, ascorbic acid, etc.), acidic polysaccharide (hyaluronic acid, chondroitin sulfate, argininic acid, etc.), sulfonic acid such as chondroitin sulfate, organic sulfonic acid including sugar ester (methane sulfonic acid, p-toluene sulfonic acid, etc.), and the like. Preferably, the salt form of the compound represented by Formula 1 may be hydrochloride.

In the present disclosure, the pharmaceutically acceptable salt may be prepared by any suitable method in the art by preparing the compound represented by Formula 1 in the form of a free base. For example, the free base may be prepared by treating a free base with inorganic acid (e.g., hydrochloric acid, bromic acid, sulfuric acid, nitric acid, methane sulfonic acid, phosphoric acid, etc.), organic acid (e.g., acetic acid, trifluoroacetic acid, maleic acid, succinic acid, mandelic acid, fumaric acid, malonic acid, pyruvic acid, oxalic acid, glycolic acid, salicylic acid, etc.), pyranosidyl acid (e.g., glucuronic acid, galacturonic acid, etc.), alpha hydroxyl acid (e.g., citric acid, tartaric acid, etc.), amino acid (e.g., aspartic acid, glutamic acid, etc.), aromatic acid (e.g., benzoic acid, cinnamic acid, etc.), sulfonic acid (e.g., p-toluene sulfonic acid, ethanol sulfonic acid, etc.), and the like.

The compound represented by Formula 1 of the present disclosure or the pharmaceutically acceptable salt thereof may be easily obtained by, for example, the method disclosed in U.S. Pat. No. 9,682,973 or in accordance with the method.

The anticancer drug as used in the present disclosure may refer to all drugs used to kill cancer cells.

In some embodiments, the anticancer drug in the second pharmaceutical composition is selected from the group consisting of a chemotherapy drug, a targeted therapy drug, an immunotherapy drug, and a hormone therapy drug. The type of anticancer drug that can be used in the composition of the present invention is not particularly limited. The anticancer drug may be selected under the general principles considered when selecting the anticancer drug, such as the type of cancer cell, the absorption rate of the anticancer drug (treatment period and the route of administration of the anticancer drug), the location of the tumor, and the size of the tumor. Specifically, the anticancer drug available in the present disclosure may be (i) chemotherapy drugs, such as alkylating agent, antimetabolites, anti-tumor antibiotics, Topoisomerase inhibitor, Mitotic inhibitors or other chemotherapy; (ii) targeted therapy drugs, such as angiogenesis inhibitor, monoclonal antibody, proteasome inhibitor, signal transduction inhibitor or new class; (iii) immunotherapy drugs, such as checkpoint inhibitor, chimeric antigen receptor (CAR) T-cell therapy or cytokine; or (iv) hormone therapy drugs, such as aromatase inhibitor, selective estrogen receptor modulators, Estrogen receptor antagonist, luteinizing hormone-releasing hormone agonist, anti-androgen or CYP17 inhibitor; but is not limited thereto.

As described above, in some embodiments, the chemotherapy drug is selected from the group consisting of an alkylating agent, an antimetabolite, an anti-tumor antibiotic, a Topoisomerase inhibitor and a Mitotic inhibitor. In some embodiments, the targeted therapy drug is selected from the group consisting of an angiogenesis inhibitor, a monoclonal antibody, a proteasome inhibitor and a signal transduction inhibitor. In some embodiments, the immunotherapy drug is selected from the group consisting of a checkpoint inhibitor, a chimeric antigen receptor (CAR) T-cell therapy and a cytokine. In some embodiments, the hormone therapy drug is selected from the group consisting of an aromatase inhibitor, a selective estrogen receptor modulators, an Estrogen receptor antagonist, a luteinizing hormone-releasing hormone agonist, an anti-androgen and a CYP17 inhibitor.

The anticancer drug available in the present disclosure may be preferably an anticancer drug belonging to the platinum class among DNA alkylating agents, or an anticancer drug belonging to the Topoisomerase inhibitor class. The anticancer drug belonging to the platinum class among DNA alkylating agents may be selected from Altretamine, Carboplatin, Cisplatin, Cyclophosphamide, Dacarbazine, Ifosfamide, Oxaliplatin, Temozolomide, and preferably Oxaliplatin, but is not limited thereto. The anticancer drug belonging to the Topoisomerase inhibitor class may be selected from Irinotecan, Irinotecan liposomal, Topotecan, Etoposide, Teniposide, and preferably Irinotecan, but is not limited thereto.

In certain embodiments, the anticancer drug is selected from irinotecan and oxaliplatin. In certain embodiments, the anticancer drug is irinotecan. In some embodiments, the anticancer drug is oxaliplatin.

The pharmaceutical composition of the present disclosure may be administered in a form in which the compound represented by Formula 1 or the pharmaceutically acceptable salt thereof is mixed with the at least one anticancer drug, or in a form in which the compound represented by Formula 1 and the at least one anticancer drug are each formulated and administered simultaneously or sequentially.

In some embodiments, the pharmaceutical compositions for co-administration are administered in which the first pharmaceutical composition and the second pharmaceutical composition are in a form of a mixture.

In some embodiments, the first pharmaceutical composition and the second pharmaceutical composition are each comprised in the form of a separate dosage form.

In some embodiments, the pharmaceutical compositions for co-administration are administered in which the first pharmaceutical composition and the second pharmaceutical composition are each formulated and administered simultaneously or sequentially.

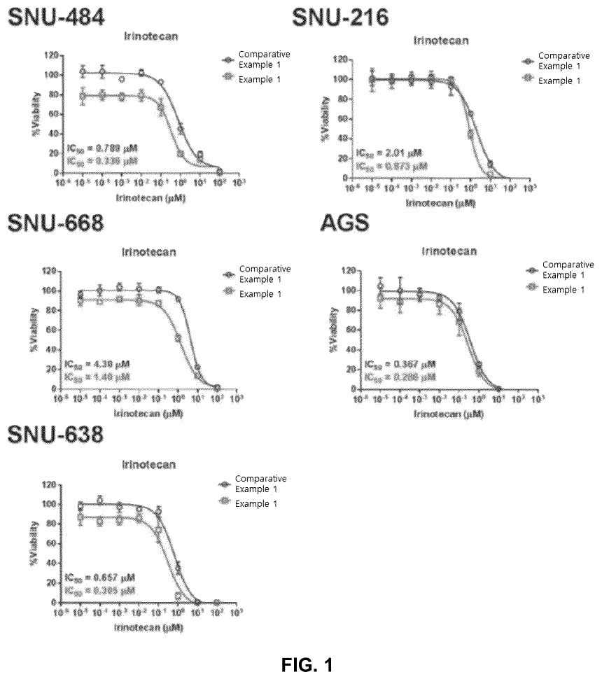

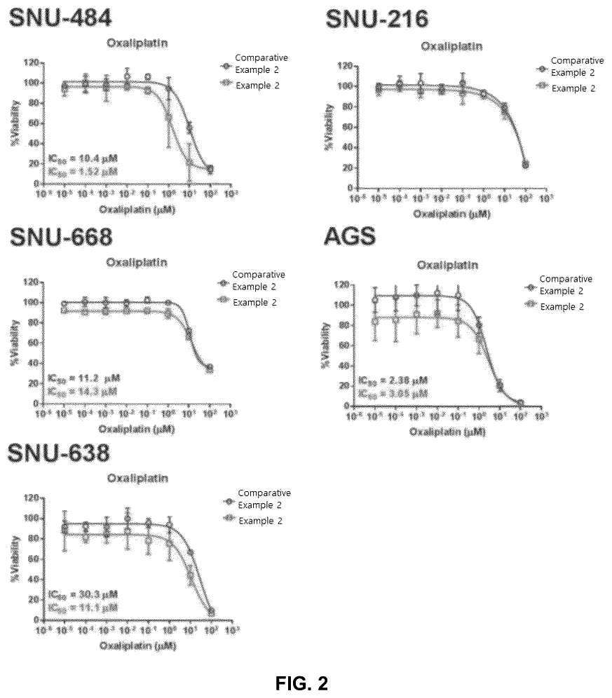

The pharmaceutical composition of the present disclosure may be in a form for simultaneous administration of two drugs that include a mixture form in which the compound represented by Formula 1 or the pharmaceutically acceptable salt thereof is mixed with the anticancer drug are mixed. The pharmaceutical composition of the present disclosure may be in a form in which the compound represented by Formula 1 or the pharmaceutically acceptable salt thereof and the anticancer drug are each formulated and administered simultaneously or sequentially. In the case of sequential administration, the order may be interchanged. According to an embodiment of the present disclosure, it is confirmed that, when the hydrochloride of the compound represented by Formula 1 is co-administered with the anticancer drug, anti-proliferative effects are exhibited in a concentration-dependent manner with respect to cancer cell lines (see FIGS. 1 and 2).

As used herein, “simultaneously” is used to mean that the two agents or compositions (e.g., pharmaceutical compositions) are administered concurrently.

As used herein, “sequentially” is used to mean that the active agents or compositions (e.g., pharmaceutical compositions) are not administered concurrently, but one after the other. Thus, administration “sequentially” may permit one agent/composition (e.g., pharmaceutical composition) to be administered within 5 minutes, 10 minutes or a matter of hours after the other provided the circulatory half-life of the first administered agent is such that they are both concurrently present in therapeutically effective amounts. The time delay between administrations of the components will vary depending on the exact nature of the components, the interaction there between, and their respective half-lives.

As used herein, “separately” is used herein to mean that the gap between administering one agent/composition (e.g., pharmaceutical composition) and the other is significant i.e. the first administered agent may no longer be present in the bloodstream in a therapeutically effective amount when the second agent is administered.

The pharmaceutical composition of the present disclosure may be used for prevention or treatment of cancer. In the present disclosure, the term “cancer” refers to cancer consisting of a mass caused by abnormal cell growth in various solid organs, such as bladder, breast, intestine, kidney, lung, liver, brain, esophagus, gallbladder, ovary, pancreas, stomach, cervix, thyroid gland, prostate, and skin.

Cancer that can be prevented or treated by the pharmaceutical composition of the present disclosure may include breast cancer, ovarian cancer, uterine endometrial cancer, small cell lung cancer, neuroendocrine tumor, biliary tract cancer, cholangiocarcinoma, uroepithelial cancer, bladder cancer, pancreatic cancer, prostate cancer, gastric cancer, esophageal cancer, colorectal cancer, thyroid cancer, hematologic cancer, cancers with homologous recombinant deficiency among aforementioned cancer types, and the like, and may preferably include gastric cancer.

In the present disclosure, the term “gastric cancer” refers to cancer occurring in the stomach, and includes adenocarcinoma occurring in glandular cells of the mucous membrane of the stomach walls and other diseases including lymphomas occurring in the lymphatic system and gastrointestinal stromal tumor occurring in the interstitial tissue.

In the present disclosure, the term “prevention” refers to any action that inhibits or delays the occurrence, spread, and recurrence of cancer by administration of the pharmaceutical composition of the present disclosure, and the term “treatment” refers to any action that improves or beneficially changes symptoms of a subject with suspected or developed cancer by administration of the pharmaceutical composition of the present disclosure.

In the present disclosure, the term “administration” refers to providing a predetermined substance to a patient by any suitable method, and regarding the administration route, the pharmaceutical composition of the present disclosure may be administered by any general route as long as it can reach a target tissue. The administration may include oral administration, intraperitoneal administration, intravenous administration, intramuscular administration, subcutaneous administration, intradermal administration, topical administration, intranasal administration, intrapulmonary administration, or intrarectal administration, but is not limited thereto. In addition, the pharmaceutical composition of the present disclosure may be administered by any device capable of transporting an active ingredient to a target cell.

Regarding preferable oral administration methods and preparations of the pharmaceutical composition of the present disclosure, each may be formulated for use in a form of powder, granule, tablet, capsule, suspension, emulsion, syrup, aerosol, and the like, according to a conventional method. Furthermore, the pharmaceutical composition of the present disclosure may be used in a form of an external preparation, a suppository, or an external preparation for skin (ointment, lotion, spray, patch, cream, powder, suspending agent, gel, gel form, etc.).

In some embodiments, the first pharmaceutical composition is to be administered once daily. In some embodiments, the first pharmaceutical composition is in an oral dosage form. In some embodiments, the first pharmaceutical composition is a tablet. In some embodiments, the first pharmaceutical composition is to be administered at a dose from about 1 mg to about 50 mg. In some embodiments, the first pharmaceutical composition is to be administered at a dose of about 5 mg, about 10 mg, about 20 mg, about 30 mg, about 40 mg, or about 50 mg per administration. In some embodiments, the first pharmaceutical composition is to be dosed at about 10 mg, about 20 mg, about 40 mg, about 80 mg, about 120 mg, or about 160 mg per administration.

Preferable parenteral administration methods and preparations of the pharmaceutical composition of the present disclosure include intravenous injections, subcutaneous injections, intradermal injections, muscular injections, drip injections, and the like. The injections may be prepared by using an aqueous solvent, such physiological saline solution and Ringer's solution, and a non-aqueous solvent, such as plant oil, high fatty acid ester (e.g., ethyl oleate, etc.), alcohol (e.g., ethanol, benzylalcohol, propyleneglycol, glycerin, etc.). The injections may include a pharmaceutical carrier, such as a stabilizer (e.g., ascorbic acid, sodium bisulfite, sodium pyrosulfite, BHA, tocopherol, EDTA, etc.) for preventing deterioration, an emulsifier, a buffer for adjusting pH, and a preservative (e.g., phenylmercuric nitrate, thimerosal, benzalkonium chloride, phenol, cresol, benzylalcohol, etc.) for inhibiting the growth of microorganisms.

In some embodiments, the second pharmaceutical composition is in an injection dosage form. In some embodiments, the second pharmaceutical composition is administered once daily. In some embodiments, the second pharmaceutical composition is to be administered intravenously. In some embodiments, the second pharmaceutical composition is to be administered continuously.

The pharmaceutical composition of the present disclosure may further include a pharmaceutically acceptable carrier. In the present disclosure, the term “pharmaceutically acceptable” refers to exhibiting non-toxic characteristics to cells or humans exposed to the composition. For use as the carrier, any material known in the art may be used without limitation, such as a buffer, a preservative, a painless agent, a solubilizer, an isotonic agent, a stabilizer, a base agent, an excipient, a lubricant, and the like.

In the present disclosure, the term “excipient” refers to a pharmaceutically acceptable inactive ingredient used in the manufacture of pharmaceutical preparations. The excipient may include, for example, a diluent, a binder, a disintegrant, a super-intense disintegrant, a glidant, a pH adjuster, a lubricant, a filler, a stabilizer, an antioxidant, a film-coating agent, and the like, but is not limited thereto. The excipient used in the present disclosure may be appropriately selected by referring to the document known in the art, for example, [Handbook of Pharmaceutical Excipients, Sixth edition, Pharmaceutical Press] which is incorporated herein by reference.

For use as a carrier, an excipient, and a diluent that can be included in the pharmaceutical composition of the present disclosure, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil may be used. When formulated, a commonly used diluent or excipient, such as a filler, an extender, a binder, a wetting agent, a disintegrant, a surfactant, and the like, may be used for preparation.

Examples of solid preparations for oral administration are a tablet, a pill, a powder, a granule, a capsule, and the like. Such solid preparations may be prepared by mixing the compound represented by Formula 1 with at least one excipient, such as starch, calcium carbonate, sucrose or lactose, or gelatin. In addition, other than a simple excipient, a lubricant such as magnesium stearate or the like may be used. Examples of liquid preparations for oral administration are a suspending agent, an oral liquid, an emulsion, a syrup, and the like. In addition to water and liquid paraffin, which are simple diluents commonly used, various excipients such as a wetting agent, a sweetening agent, a fragrance, a preservative, and the like may be included.

Preparations for parenteral administration may include a sterile solution, a nonaqueous solvent, a suspending agent, an emulsion, a freeze-dried agent, a suppository, and the like. As a nonaqueous solvent and a suspending agent, propylene glycol, polyethylene glycol, vegetable oils such as olive oil, and injectable ester such as ethyl oleate may be used. As a base agent for suppository, witepsol, macrogol, tween 61, cacao butter, laurinum, glycerogelatin, or the like may be used.

Meanwhile, with a pharmaceutically effective amount of the pharmaceutical composition of the present disclosure, in mammals including humans, the pharmaceutical composition of the present disclosure may be used alone or in combination with other active ingredients included in the pharmaceutical composition or with a kit, a product, or a concomitant agent, thereby preventing, removing, or reducing harmful effects of cancer.

In the present disclosure, the term “pharmaceutically effective amount” refers to an amount sufficient to treat a disease with a reasonable benefit/risk ratio applicable to medical treatment, and the level of “therapeutically effective amount” may be determined by factors, which includes a patient's health condition, a cancer type, severity, drug activity, drug sensitivity, an administration method, an administration time, an administration route and an excretion rate, a period of treatment, combination or drugs simultaneously used, and other factors well known in the medical field. It is understood that a dose administered in the present disclosure may be lower for each compound in the composition relative to “therapeutically effective amount” defined for each compound used alone or in combination with treatments other than the combined therapy described above. In particular, the pharmaceutical composition, the kit, the product, and the concomitant agent of the present disclosure may reduce tumor development, reduce tumor burden, cause tumor regression in a mammalian host, and/or prevent occurrence of metastasis or cancer. In the cancer treatment, the pharmaceutical composition of the present disclosure may be administered in a therapeutically effective amount.

In an embodiment, a daily dose of the compound represented by Formula 1 of the present disclosure may be about 1 mg to about 400 mg, preferably, about 2 mg to about 240 mg, more preferably, about 5 mg to about 160 mg, and even more preferably, about 5 mg to about 120 mg. In addition, a daily dose of irinotecan which is the anticancer drug of the present disclosure may be about 10 mg/m² to about 350 mg/m², preferably, about 25 mg/m² to about 100 mg/m², and more preferably, about 25 mg/m² to about 75 mg/m². In some embodiments, the anticancer drug (e.g., irinotecan) is to be intravenously dosed at about 30 mg/m² to about 120 mg/m². In some embodiments, the anticancer drug (e.g., irinotecan) is to be dosed at about 50 mg/m², about 75 mg/m², or about 100 mg/m². In some embodiments, the anticancer drug (e.g., irinotecan) is to be intravenously dosed at about 125 mg/m², 180 mg/m², or about 350 mg/m². In some embodiments, the second pharmaceutical composition is to be intravenously dosed at about 30 mg/m² to about 120 mg/m². In some embodiments, the second pharmaceutical composition is to be dosed at about 50 mg/m², about 75 mg/m², or about 100 mg/m². However, due to the nature of cancer which is a target disease of the pharmaceutical composition of the present disclosure, a dose of each ingredient may vary greatly depending on the conditions described above, and is not limited to the embodiments above.

As used herein, the term “about” or “approximately” refers to a ±10% variation from the recited quantitative value (and includes the recited quantitative value itself) unless otherwise indicated or inferred from the context. For example, unless otherwise stated or inferred from the context, a dose of about 100 mg/m² indicates a dose range of 100±10% mg/m², i.e., from 90 mg/m² to 110 mg/m², inclusive.

Regarding the pharmaceutical composition for co-administration of the present disclosure, a suitable administration cycle of (i) the compound represented by Formula 1 or the pharmaceutically acceptable salt thereof and (ii) the anticancer drug may be determined according to the dose. In the present disclosure, (i) the compound represented by Formula 1 or the pharmaceutically acceptable salt thereof may be administered once a day to once a week, and preferably, once a day, and (ii) the anticancer drug may be administered once a day to once a week, and preferably, once a week.

In an embodiment of the present disclosure, one cycle of administration of the pharmaceutical composition for co-administration of the present disclosure may be 14 days to 21 days, but is not limited thereto. In an embodiment of the present disclosure, when the (one) cycle of the administration is 14 days, (i) from the 1st day to the 7th day, from the 1st day to the 5th day, or from 2nd day to the 6th day, the compound represented by Formula 1 or the pharmaceutically acceptable salt may be orally administered once a day or administered by injection once a day, and (ii) on the 1st day or from the 2nd day to the 6th day, the anticancer drug may be administered by injection once a day.

In an embodiment of the present disclosure, when the (one) cycle of the administration is 21 days, (i) from the 1st day to the 7th day, from the 1st day to the 5th day or from 2nd day to the 6th day, the compound represented by Formula 1 or the pharmaceutically acceptable salt may be orally administered once a day or administered by injection once a day, and (ii) on the 1st day, or from the 2nd day to the 6th day, the anticancer drug may be administered by injection once a day.

In some embodiments, the first pharmaceutical composition is to be dosed as a cycle, wherein the cycle comprises administering the first pharmaceutical composition for about 14 days or about 21 days. For example, in some embodiments, the first pharmaceutical composition is administered to a subject in need thereof for 1, 2, 3, 4, 5, 6, 7, 8, 9, 10, 11, 12, 13, or 14 days.

In some embodiments, the second pharmaceutical composition is to be dosed as a cycle, wherein the cycle comprises administering the second pharmaceutical composition for about 14 days or about 21 days. In some embodiments, the second pharmaceutical composition is to be dosed as a cycle, wherein the cycle is repeated at least once. For example, in some embodiments, the second pharmaceutical composition is administered to a subject in need thereof for 1, 2, 3, 4, 5, 6, 7, 8, 9, 10, 11, 12, 13, or 14 days.

In some embodiments, in one cycle, the first pharmaceutical composition is to be administered from the 1st day to the 7th day, from the 1st day to the 5th day, or from 2nd day to the 6th day; and the second pharmaceutical composition is to be administered on the 1st day, or from 2nd to 6th day, wherein the first pharmaceutical composition is to be administered once daily, the second pharmaceutical composition is to be administered once daily, and the one cycle has a duration of 14 days or 21 days. In some embodiments, in one cycle, the first pharmaceutical composition containing 40 mg, 20 mg, or 10 mg of the compound of Formula 1, or a pharmaceutically acceptable salt thereof, is to be administered once daily, and the second pharmaceutical composition is to be administered once daily at a intravenously dose of 100 mg/m².

The pharmaceutical composition for co-administration of the present disclosure may be administered as an individual therapeutic agent, may be administered again in combination with other therapeutic agents, may be administered sequentially or simultaneously with a conventional therapeutic agent, and may be administered once or multiple times. In consideration of all of the factors described above, it is important to administer an amount capable of obtaining the maximum effect with a minimum amount without side effects.

In the present disclosure, the term “kit”, “product”, or “concomitant agent” specifically defines “kit of components” in the sense that the compound represented by Formula 1 as defined above may be administered independently or used in combination with a unique amount of a fixed combination different from a combined anticancer drug, and that is, may be administered at the same time or at different times. Components of the component kit may be then administered, for example, at the same time or in chronological order, i.e., at different times. In other words, any component of the component kit may be administered at the same time or at different time intervals. A ratio of the total amounts of the compound represented by Formula 1 and the concomitant anticancer drug administered in the concomitant preparations may vary. The combined anticancer drug may be administered by the same or different route. In some embodiments, the first pharmaceutical composition is present in a single dosage form and the second pharmaceutical composition is present in a separate dosage form. In some embodiments, the first pharmaceutical composition is to be administered simultaneously, separately, or sequentially.

In the present disclosure, the term “treatment” refers to curative, symptomatic, and prophylactic treatment. The pharmaceutical composition, the kit, the product, and the concomitant agent of the present disclosure may be used in humans with cancer or tumor at an early stage or latter stages of cancer progression.

The pharmaceutical composition, the kit, the product, and the concomitant agent of the present disclosure may not necessarily treat a patient with cancer, but may improve a patient's condition by delaying or slowing the progression or preventing further progression of the disease.

Throughout the specification of the present disclosure, when the term “cancer treatment” is mentioned in connection with the pharmaceutical composition of the present disclosure, the cancer treatment may refer to: (a) as a method of treating cancer, the method including administering the pharmaceutical composition of the present disclosure to a target in need of such treatment; (b) use of the pharmaceutical composition of the present disclosure for treatment of cancer; (c) use of the pharmaceutical composition of the present disclosure for preparation of a pharmaceutical for treatment of cancer; and/or (d) the pharmaceutical composition of the present disclosure for use in treatment of cancer.

Another aspect of the present disclosure provides a method of preventing or treating cancer, the method including administering the pharmaceutical composition for co-administration to a subject in need thereof, wherein the pharmaceutical composition includes (i) the compound represented by Formula 1 or the pharmaceutically acceptable salt thereof and (ii) at least one anticancer drug.

Contemplated herein, on in embodiments, are methods of preventing or treating cancer in a subject in need thereof, the methods comprising administering a pharmaceutical composition described herein.

In an aspect, the present disclosure provides a method of treating cancer in a subject in need thereof, wherein the method comprises administering to the subject (i) a first pharmaceutical composition comprising a compound represented by Formula 1 or a pharmaceutically acceptable salt thereof;

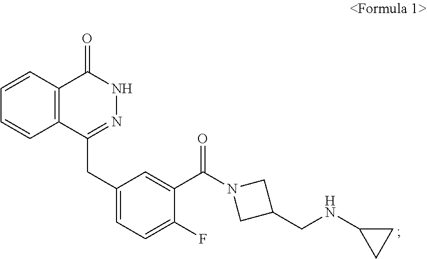 [SMILES: O=C(c1cc(Cc2n[nH]c(=O)c3ccccc23)ccc1F)N1CC(CNC2CC2)C1]

and
(ii) a second pharmaceutical composition comprising an anticancer drug. In some embodiments, the cancer is gastric cancer. In some embodiments, the cancer is advanced gastric cancer. In some embodiments, the anticancer drug is selected from irinotecan and oxaliplatin. In some embodiments, the anticancer drug is irinotecan. In some embodiments, the anticancer drug is oxaliplatin. In some embodiments, the pharmaceutically acceptable salt is hydrochloride.

Another aspect of the present disclosure provides a use of a compound represented by Formula 1 or a pharmaceutically acceptable salt thereof for the manufacture of a first medicament and use of an anticancer drug for the manufacture of a second medicament, wherein the first medicament and the second medicament are for preventing or treating a cancer in a subject in need thereof. In some embodiments, the cancer is gastric cancer. In some embodiments, the cancer is advanced gastric cancer. In some embodiments, the anticancer drug is selected from irinotecan and oxaliplatin. In some embodiments, the anticancer drug is irinotecan. In some embodiments, the anticancer drug is oxaliplatin. In some embodiments, the pharmaceutically acceptable salt is hydrochloride.

Yet another aspect of the present disclosure provides a use of a first pharmaceutical composition comprising a compound represented by Formula 1 or a pharmaceutically acceptable salt thereof for the manufacture of a first medicament and use of a second pharmaceutical composition comprising an anticancer drug for the manufacture of a second medicament, wherein the first medicament and the second medicament are for preventing or treating a cancer in a subject in need thereof. In some embodiments, the cancer is gastric cancer. In some embodiments, the cancer is advanced gastric cancer. In some embodiments, the anticancer drug is selected from irinotecan and oxaliplatin. In some embodiments, the anticancer drug is irinotecan. In some embodiments, the anticancer drug is oxaliplatin. In some embodiments, the pharmaceutically acceptable salt is hydrochloride.

In some embodiments, the anticancer drug is selected from the group consisting of a chemotherapy drug, a targeted therapy drug, a immunotherapy drug, and a hormone therapy drug. In some embodiments, the chemotherapy drug is selected from the group consisting of an alkylating agent, an antimetabolite, an anti-tumor antibiotic, a Topoisomerase inhibitor and a Mitotic inhibitor. In some embodiments, the targeted therapy drug is selected from the group consisting of an angiogenesis inhibitor, a monoclonal antibody, a proteasome inhibitor and a signal transduction inhibitor. In some embodiments, the immunotherapy drug is selected from the group consisting of a checkpoint inhibitor, a chimeric antigen receptor (CAR) T-cell therapy and a cytokine. In some embodiments, the hormone therapy drug is selected from the group consisting of an aromatase inhibitor, a selective estrogen receptor modulators, an Estrogen receptor antagonist, a luteinizing hormone-releasing hormone agonist, an anti-androgen and a CYP17 inhibitor. In some embodiments, the anticancer drug is selected from irinotecan and oxaliplatin. In some embodiments, the anticancer drug is irinotecan. In some embodiments, the anticancer drug is oxaliplatin. In some embodiments, the pharmaceutically acceptable salt is hydrochloride.

In some embodiments, the method or use comprises administering the first pharmaceutical composition and the second pharmaceutical composition in a form of a mixture. In some embodiments, the method or use comprises administering the first pharmaceutical composition and the second pharmaceutical composition in separate formulations, wherein the first pharmaceutical composition and second pharmaceutical composition are administered simultaneously or sequentially.

In some embodiments, the method or use comprises administering the first pharmaceutical composition and the second pharmaceutical composition each in the form of a separate dosage form. In some embodiments, the method or use comprises administering the first pharmaceutical composition in an oral dosage form, and the second pharmaceutical composition in an injection dosage form. In some embodiments, the method or use comprises administering the first pharmaceutical composition in a tablet, and the second pharmaceutical composition intravenously.

In some embodiments, the method or use comprises administering the first pharmaceutical composition and the second pharmaceutical composition each once daily. In some embodiments, the method or use comprises administering the first pharmaceutical composition is to be dosed at about 1 mg to about 180 mg. In some embodiments, the method or use comprises administering the first pharmaceutical composition is to be dosed at about 5 mg to about 170 mg. In some embodiments, the method or use comprises administering the first pharmaceutical composition is to be dosed at about 10 mg to about 160 mg. In some embodiments, the method or use comprises administering the first pharmaceutical composition at a dose of about 10 mg, about 20 mg, about 40 mg, about 80 mg, about 120 mg, or about 160 mg per administration. In some embodiments, the first pharmaceutical composition comprises about 1 mg to about 50 mg of the compound represented by Formula 1. In some embodiments, the first pharmaceutical composition comprises about 5 mg to about 40 mg of the compound represented by Formula 1. In some embodiments, the first pharmaceutical composition comprises about 5 mg, about 10 mg, about 20 mg, or about 40 mg of the compound represented by Formula 1. In some embodiments, the first pharmaceutical composition is administered to a subject in need thereof with at least 8 ounces of water.

In some embodiments, the method or use comprises administering the second pharmaceutical composition is to be intravenously dosed at about 30 mg/m² to about 120 mg/m². In some embodiments, the method or use comprises administering the second pharmaceutical composition is to be intravenously dosed at about 50 mg/m², about 75 mg/m², or about 100 mg/m². In some embodiments, the method or use comprises administering the first pharmaceutical composition and the second pharmaceutical composition in one cycle, wherein the one cycle has a duration of about 14 or 21 days. In some embodiments, the cycle is repeated at least once. In some embodiments, the second pharmaceutical composition comprises an anticancer drug (e.g., irinotecan) at a concentration of about 20 mg/mL. In some embodiments, the second pharmaceutical composition comprises 5% dextrose or 0.9% sodium chloride. In some embodiments, the anticancer drug (e.g., irinotecan) is administered for about 90 minutes. In some embodiments, the anticancer drug (e.g., irinotecan) is administered for more than about 90 minutes.

In some embodiments, the method or use comprises administering the first pharmaceutical composition at a dosage of about 40 mg, and intravenously administering the second pharmaceutical composition at a dosage of about 50 mg/m². In some embodiments, the method or use comprises administering the first pharmaceutical composition at a dosage of about 40 mg, and intravenously administering the second pharmaceutical composition at a dosage of about 75 mg/m². In some embodiments, the method or use comprises administering the first pharmaceutical composition at a dosage of about 10 mg, and intravenously administering the second pharmaceutical composition at a dosage of about 100 mg/m². In some embodiments, the method or use comprises administering the first pharmaceutical composition at a dosage of about 20 mg, and intravenously administering the second pharmaceutical composition at a dosage of about 100 mg/m². In some embodiments, the method or use comprises administering the first pharmaceutical composition at a dosage of about 40 mg, and intravenously administering the second pharmaceutical composition at a dosage of about 100 mg/m². In some embodiments, the method or use comprises administering the first pharmaceutical composition at a dosage of about 80 mg, and intravenously administering the second pharmaceutical composition at a dosage of about 100 mg/m². In some embodiments, the method or use comprises administering the first pharmaceutical composition at a dosage of about 120 mg, and intravenously administering the second pharmaceutical composition at a dosage of about 100 mg/m².

In some embodiments, the method or use comprises administering the first pharmaceutical composition at a dosage of about 40 mg once daily, and intravenously administering the second pharmaceutical composition at a dosage of about 50 mg/m² once daily. In some embodiments, the method or use comprises administering the first pharmaceutical composition at a dosage of about 40 mg once daily, and intravenously administering the second pharmaceutical composition at a dosage of about 75 mg/m² once daily. In some embodiments, the method or use comprises administering the first pharmaceutical composition at a dosage of about 10 mg once daily, and intravenously administering the second pharmaceutical composition at a dosage of about 100 mg/m² once daily. In some embodiments, the method or use comprises administering the first pharmaceutical composition at a dosage of about 20 mg once daily, and intravenously administering the second pharmaceutical composition at a dosage of about 100 mg/m² once daily. In some embodiments, the method or use comprises administering the first pharmaceutical composition at a dosage of about 40 mg once daily, and intravenously administering the second pharmaceutical composition at a dosage of about 100 mg/m² once daily. In some embodiments, the method or use comprises administering the first pharmaceutical composition at a dosage of about 80 mg once daily, and intravenously administering the second pharmaceutical composition at a dosage of about 100 mg/m² once daily. In some embodiments, the method or use comprises administering the first pharmaceutical composition at a dosage of about 120 mg once daily, and intravenously administering the second pharmaceutical composition at a dosage of about 100 mg/m² once daily.

In some embodiments, the present disclosure provides a method of treating cancer, wherein the method comprises administering to a subject in need thereof (i) a first pharmaceutical composition comprising a compound represented by Formula 1 or a pharmaceutically acceptable salt thereof;

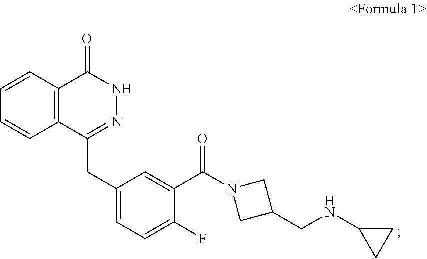 [SMILES: O=C(c1cc(Cc2n[nH]c(=O)c3ccccc23)ccc1F)N1CC(CNC2CC2)C1]

and
(ii) a second pharmaceutical composition comprising irinotecan. In some embodiments, the cancer is gastric cancer. In some embodiments, the cancer is advanced gastric cancer.

In some embodiments, the present disclosure provides a method of treating cancer, wherein the method comprises administering to a subject in need thereof (i) a first pharmaceutical composition comprising a compound represented by Formula 1 or a pharmaceutically acceptable salt thereof;

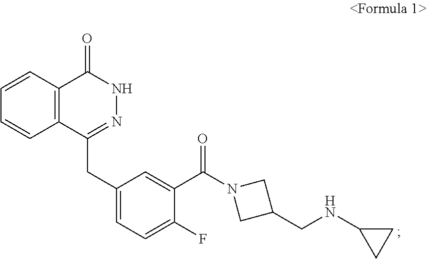 [SMILES: O=C(c1cc(Cc2n[nH]c(=O)c3ccccc23)ccc1F)N1CC(CNC2CC2)C1]

and
(ii) a second pharmaceutical composition comprising oxaliplatin. In some embodiments, the cancer is gastric cancer. In some embodiments, the cancer is advanced gastric cancer.

In the present disclosure, the term “subject” refers to all animals including humans that have developed or can develop cancer, and the cancer may be effectively prevented or treated by administering the pharmaceutical composition of the present disclosure to a subject. The pharmaceutical composition of the present disclosure may be administered in combination of the existing therapeutic agent.

The pharmaceutical composition of the present disclosure may be used in combination with other therapies known for the treatment of diseases. Other therapies may include chemotherapy, radiation therapy, hormone therapy, bone marrow transplantation, stem-cell replacement therapy, other biological therapies, immunotherapy, and the like, but are not limited thereto.

As described herein, therapeutic efficacy of the combination of the pharmaceutical compositions of the disclosure can be determined by various endpoints. In some embodiments, the therapeutic efficacy of the treatment is determined by assessing improvement based on the objective response rate (ORR), progression-free survival (PFS), disease control rate (DCR), duration of response (DOR), time to response (TTR), best overall response (BOR), time to progression (TTP), or overall survival (OS) as evaluated by Response Evaluation Criteria in Solid Tumors (RECIST).

Moreover, therapeutic efficacy of the combination of the pharmaceutical compositions of the disclosure can be determined by a reduction in tumor volume. For example, the therapeutic efficacy of the combination can be determined by a reduction in volume growth of the tumor after administration of the combination when compared with that of the anticancer drug alone. The therapeutic efficacy of the combination can also be determined by the inhibitory effect on the proliferation of tumor cells. For example, the therapeutic efficacy of the combination can be determined by the inhibitory effect on the proliferation of tumor cells after administration of the combination when compared with that of the anticancer drug alone.

### Advantageous Effects of Disclosure

It is confirmed that a compound represented by Formula 1 of the present disclosure or a pharmaceutically acceptable salt thereof shows a synergistic complementary effect when co-administered with a known anticancer drug, as compared with administration of an anticancer drug alone, thereby exhibiting excellent anticancer activity. When the compound represented by Formula 1 of the present disclosure or the pharmaceutically acceptable salt thereof is co-administered with an anticancer drug, specifically irinotecan or oxaliplatin, the same or better anticancer effect can be obtained even at a lower dose than the previously known anticancer drug, and excellent effects can be even obtained in cancers that have been limited by the existing treatments. In addition, effects of reducing dose-dependent side effects may be obtained. The pharmaceutical composition for co-administration of the present disclosure is remarkably useful as an anticancer drug by exhibiting considerably synergistic effects in prevention or treatment of cancer, especially even solid cancer such as gastric cancer, by specially combining the compound represented by Formula 1 or the pharmaceutically acceptable salt thereof (first active ingredient) and the anticancer drug (second active ingredient).

### DETAILED DESCRIPTION OF THE DRAWINGS

FIG. 1 is a graph showing cell viability compared in a case where hydrochloride of a compound represented by Formula 1 is co-administered with irinotecan with a case where irinotecan is administered alone.

FIG. 2 is a graph showing cell viability compared in a case where hydrochloride of a compound represented by Formula 1 is co-administered with oxaliplatin with a case where oxaliplatin is administered alone.

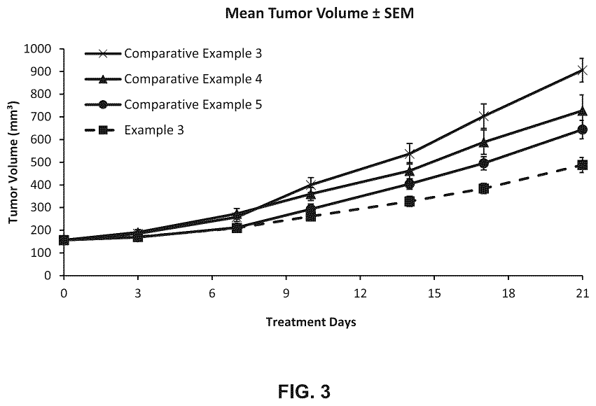

FIG. 3 is a graph showing effects on tumor volume compared in a case where hydrochloride of the compound of Formula 1 is administered alone with a case where hydrochloride of the compound of Formula 1 is co-administered with irinotecan, in a subcutaneous MKN45 human gastric cancer xenograft model (mean±SEM).

### DETAILED DESCRIPTION

Hereinafter, the present disclosure will be described in detail with reference to Examples below. However, Examples below are for illustrating the present disclosure, and the present disclosure is not limited thereto.

To evaluate preventive and therapeutic effects of the pharmaceutical composition of the present disclosure in prevention and treatment of cancer, experiments below were carried out as follow.

### Experimental Example 1

### 1.1. Preparation and Treatment of DMSO Stock Solution

Hydrochloride of a compound represented by Formula 1 (M.W. 442.92, hereinafter, referred to as ‘hydrochloride compound of Formula 1’) was obtained from Ildong Pharmaceutical. Irinotecan hydrochloride (M.W. 623.14) was obtained from Sigma Aldrich.

Each of these drugs was dissolved in 100% DMSO to prepare a 10 mM stock solution, which was then divided into single doses and stored at −20° C.

The 10 mM stock solution containing hydrochloride compound of Formula 1 and irinotecan was serially diluted in DMSO at a ratio of 1/10 to prepare a stock solution for each treatment concentration. Here, DMSO was used in a final content not exceeding 1.1% to treat the cells.

As cell lines, five human-derived gastric cancer cell lines (i.e., AGS, SNU-668, SNU-216, SNU-484, and SNU-638) were used. The source of the cell lines is the Korean Cell Line Bank (KCLB). As a medium, a complete growth medium consisting of 90% RPMI1640 and 10% heat-inactivated fetal bovine serum was used.

### 1.2. Thawing and Subculturing of Frozen Cells

Frozen cells were thawed in a water bath at 37° C., suspended in a culture medium, and then centrifuged at 1,200 rpm for 2 minutes. The precipitated cells were suspended in a culture medium, spread on a culture dish, and cultured at 37° C. in 5% CO₂. Reflecting the growth rate of each cell, the cells were cultured periodically at intervals of 3 days to 5 days.

### 1.3. Cell Plating and Drug Treatment

To remove the cells from the culture dish, the cells were treated with trypsine and allowed for a reaction in an incubator at 37° C. in 5% CO₂. The cells collected after the completion of the reaction were suspended in a culture medium, and centrifuged at 1,200 rpm for 3 minutes. The cell suspension was put into a hemocytometer to calculate the number of cells per unit volume. According to the conditions set for each cell line, the cell suspension was plated into a 96-well plate and then stabilized for 24 hours at 37° C. in 5% CO₂.

The 10 mM stock solution containing irinotecan was serially diluted in DMSO at a ratio of 1/10 to prepare a 100× stock solution for each treatment concentration, and a 1000× stock solution containing 1 mM hydrochloride compound of Formula 1 was prepared, so as to prepare a stock solution with a final treatment concentration of 1 μM. To minimize the effect of the solvent on the cells, each well contained 1.1% DMSO. The stock solution of each concentration was added to a culture medium to have a 10× concentration, and then the cells were treated at a 1× concentration. Each cell line was treated with irinotecan alone or in combination of hydrochloride compound of Formula 1 and irinotecan, each for 5 days.

### 1.4. MTT Assay

After the treatment of each drug, 100 μL of 3-(4,5-dimethylthiazol-2-yl)-2,5-diphenyltetrazolium bromide solution (Sigma Aldrich) was added to each well, the plate was cultured at 37° C. for 2 hours, and the culture medium was removed. After formazan crystals were dissolved in 100 μL of DMSO, a Synergy H4 microplate reader (BioTek) was used to measure absorbance of each cell at 570 nm.

### 1.5. Data Analysis

The absorbance was calculated as activity expressed in percent at each concentration of the drugs, as compared with a negative control group (100%), which is the group including a solvent (DMSO) only. IC₅₀ was calculated by nonlinear regression analysis in 3 or 4 parameter fits (GraphPad Prism version 7.04).

According to the MTT assay, the sensitivity was confirmed through the viability of each cell line in irinotecan with or without 1 μM of hydrochloride compound of Formula 1, and results thereof are shown in FIG. 1. As shown in FIG. 1, the administration of irinotecan alone exhibited an effect of cell death, whereas the co-administration of irinotecan with the compound of Formula 1 resulted in a higher cell death rate. Accordingly, it was confirmed that the inhibitory effect on cancer growth obtained by such co-administration increased in a dose-dependent manner. The calculated IC₅₀ values are shown in Table 1.

TABLE 1

| | IC₅₀ value (μM) | | | | |
|---|---|---|---|---|---|
| Drug administered | SNU-484 | SNU-216 | SNU-668 | AGS | SNU-638 |
| Example 1 | 0.336 | 0.873 | 1.40 | 0.286 | 0.305 |
| (administration of | | | | | |
| hydrochloride | | | | | |
| compound of | | | | | |
| Formula 1 and | | | | | |
| irinotecan) | | | | | |
| Comparative | 0.789 | 2.01 | 4.30 | 0.367 | 0.657 |
| Example 1 | | | | | |
| (administration | | | | | |
| of irinotecan | | | | | |
| alone) | | | | | |
| The fold | 2.3 | 2.3 | 3.1 | 1.3 | 2.2 |
| change (ratio) | | | | | |

The fold change (ratio)=IC₅₀ in the presence of DMSO/IC₅₀ in the presence of 1 μM hydrochloride of Compound 1

Referring to Table 1, the IC₅₀ values of the five cell lines in Example 1 were found to be 1.3 times to 3.1 times lower than those in Comparative Example 1. Accordingly, it was confirmed that, in the experiment using the gastric cancer cell lines, the pharmaceutical composition of the present disclosure exhibited excellent inhibitory effect on the proliferation of tumor cells, compared to the administration of the anticancer drug alone.

### Experimental Example 2

### 2.1. Preparation and Treatment of DMSO Stock Solution

Hydrochloride of the compound represented by Formula 1 (M.W. 442.92, hereinafter, referred to as ‘hydrochloride compound of Formula 1’) was obtained from Ildong Pharmaceutical. Oxaliplatin (M.W. 397.29) was obtained from Sigma Aldrich.

Each of these drugs was dissolved in 100% DMSO to prepare a 10 mM stock solution, which was then divided into single doses and stored at −20° C.

The 10 mM stock solution containing hydrochloride compound of Formula 1 and oxaliplatin was serially diluted in DMSO at a ratio of 1/10 to prepare a stock solution for each treatment concentration. Here, DMSO was used in a final content not exceeding 1.1% to treat the cells.

As cell lines, five human-derived gastric cancer cell lines (i.e., AGS, SNU-668, SNU-216, SNU-484, and SNU-638) were used. The source of the cell lines is the KCLB. As a medium, a complete growth medium consisting of 90% RPMI1640 and 10% heat-inactivated fetal bovine serum was used.

### 2.2. Thawing and Subculturing of Frozen Cells

Frozen cells were thawed in a water bath at 37° C., suspended in a culture medium, and then centrifuged at 1,200 rpm for 2 minutes. The precipitated cells were suspended in a culture medium, spread on a culture dish, and cultured at 37° C. in 5% CO₂. Reflecting the growth rate of each cell, the cells were cultured periodically at intervals of 3 days to 5 days.

### 2.3. Cell Plating and Drug Treatment

To remove the cells from the culture dish, the cells were treated with trypsin and allowed for a reaction in an incubator at 37° C. in 5% CO₂. The cells collected after the completion of the reaction were suspended in a culture medium, and centrifuged at 1,200 rpm for 3 minutes. The cell suspension was put into a hemocytometer to calculate the number of cells per unit volume. According to the conditions set for each cell line, the cell suspension was plated into a 96-well plate and then stabilized for 24 hours at 37° C. in 5% CO₂.

The 10 mM stock solution containing oxaliplatin was serially diluted in DMSO at a ratio of 1/10 to prepare a 100× stock solution for each treatment concentration, and a 1000× stock solution containing 1 mM hydrochloride compound of Formula 1 was prepared, so as to prepare a stock solution with a final treatment concentration of 1 μM. To minimize the effect of the solvent on the cells, each well contained 1.1% DMSO. The stock solution of each concentration was added to a culture medium to have a 10× concentration, and then the cells were treated at a 1× concentration. Each cell line was treated with oxaliplatin alone or in combination of hydrochloride compound of Formula 1 and oxaliplatin, each for 5 days.

### 2.4. MTT Assay

After the treatment of each drug, 100 μL of 3-(4,5-dimethylthiazol-2-yl)-2,5-diphenyltetrazolium bromide solution (Sigma Aldrich) was added to each well, the plate was cultured at 37° C. for 2 hours, and the culture medium was removed. After formazan crystals were dissolved in 100 μL of DMSO, a Synergy H4 microplate reader (BioTek) was used to measure absorbance of each cell at 570 nm.

The absorbance was calculated as activity expressed in percent at each concentration of each drug, as compared with a negative control group (100%), which is the group including a solvent (DMSO) only. IC₅₀ was calculated by nonlinear regression analysis in 3 or 4 parameter fits (GraphPad Prism 7.04).

According to the MTT assay, the sensitivity was confirmed through the viability of each cell line in oxaliplatin with or without 1 μM of hydrochloride compound of Formula 1, and results thereof are shown in FIG. 2. As shown in FIG. 2, the administration of oxaliplatin alone exhibited an effect of cell death, whereas the co-administration of oxaliplatin with compound of Formula 1 resulted in a higher cell death rate. Accordingly, it was confirmed that the inhibitory effect on cancer growth obtained by such co-administration increased in a dose-dependent manner. The calculated IC₅₀ values are shown in Table 2.

TABLE 2

| | IC₅₀ value (μM) | | | | |
|---|---|---|---|---|---|
| Drug administered | SNU-484 | SNU-216 | SNU-668 | AGS | SNU-638 |
| Example 2 | 1.52 | (n.a.) | 14.3 | 3.05 | 11.1 |
| (administration of | | | | | |
| hydrochloride | | | | | |
| compound | | | | | |
| of Formula | | | | | |
| 1 and oxaliplatin) | | | | | |
| Comparative | 10.4 | (n.a.) | 11.2 | 2.38 | 30.3 |
| Example 2 | | | | | |
| (administration of | | | | | |
| oxaliplatin alone) | | | | | |
| The fold | 6.8 | (n.a.) | 0.8 | 0.8 | 2.7 |
| change (ratio) | | | | | |

The fold change (ratio)=IC₅₀ in the presence of DMSO/IC₅₀ in the presence of 1 μM hydrochloride of Compound 1

Referring to Table 2, the IC₅₀ values of the specific cell lines in Example 2 were found to be 2.7 times to 6.8 times lower than those in Comparative Example 2. Accordingly, it was confirmed that, in the experiment using the gastric cancer cell lines, the pharmaceutical composition of the present disclosure exhibited excellent inhibitory effect on the proliferation of tumor cells, compared to the administration of the anticancer drug alone.

### Experimental Example 3

### 3.1. Cell Culture

MKN45 tumor cells were cultured in RPMI1640 supplemented with 10% fetal bovine serum at 37° C. in 5% CO₂. The cells were harvested before tumor transplantation was performed, and then counted using a cell counter.

### 3.2. Tumor Transplantation

To perform tumor transplantation, 0.1 ml of PBS tumor suspension (3×10⁶ of MKN45 tumor cells) were subcutaneously inoculated into the upper right flank of each mouse.

### 3.3. Randomization

Randomization was performed when the average tumor size of the mouse reached about 150 mm³. A total of 40 mice were used for the test, and 10 mice per group were assigned to 4 groups as shown in Table 3 (Example 3 and Comparative Examples 3 to 5). Randomization was performed based on a “matched distribution” method (Study Director™ software, version 3.1.399.19). Here, the grouping date was marked as 0.

TABLE 3

| | | | Level | Solution | Volumn | | Frequency and |
|---|---|---|---|---|---|---|---|
| | Number | Drug | administered | administered | administered | | duration of |
| Division | of mice | administered | (mg/kg) | (mg/mL) | (mL/kg) | ROA | administration |
| Example 3 | 10 | Hydrochloride | 2 | 0.2 | 10 | p.o. | QD × 22 days |
| | | compound of | | | | | |
| | | Formula 1 | | | | | |
| | | Irinotecan | 5 | 1 | 5 | i.v. | QW × 4 times |
| Comparative | 10 | Vehicle | — | — | 10 | p.o. | QD × 22 days |
| Example 3 | | | | | | | |
| Comparative | 10 | Hydrochloride | 2 | 0.2 | 10 | p.o. | QD × 22 days |
| Example 4 | | compound of | | | | | |
| | | Formula 1 | | | | | |
| Comparative | 10 | Irinotecan | 5 | 1 | 5 | i.v. | QW × 4 times |
| Example 5 | | | | | | | |

### 3.4. Observation and Collection

After tumor inoculation, the morbidity and mortality of the animals were checked every day. During routine monitoring, effects of tumor growth and treatment in the animals on mobility, food and water consumption, weight gain/loss (weight was measured twice a week after randomization), eye/hair lubrication, and behavior such as other abnormalities were confirmed. Mortality and observed clinical symptoms were recorded in detail for each animal.

Tumor volume was measured using a calipers twice a week after randomization, and was calculated according to the following formula:

$$V = ( Length \times Width \times Width ) / 2$$

V (mm³) refers to a volume of tumor, L refers to a length of tumor (the longest size of tumor), and W refers to a width of tumor (longest size of tumor perpendicular to L). In addition to the measurement of tumor and weight, administration was performed in a Laminar Flow Cabinet.

The weight and tumor volume were measured using a Study Director™ software (version 3.1.399.19). As a result of the measurement in all groups, no significant weight loss was observed.

### 3.5. Drug Formulations for Co-Administration

Formulations of drug for co-administration are as shown in Table 4.

TABLE 4

| | Level | Con- | | | Preparation |
|---|---|---|---|---|---|
| | admin- | cen- | | Formu- | frequency |
| Drug | istered | tration | | lation | and |
| administered | (mg/kg) | (mg/mL) | Preparation | type | storage |
| Vehicle | — | — | Sterile distilled | Solution | |
| | | | water | | |
| Hydrochloride | 2 | 0.2 | 0.24 mL of | Solution | Fresh |
| compound of | | | 5 mg/mL | | before |
| Formula 1 | | | of a solution | | adminis- |
| | | | containing the | | tration |
| | | | hydrochloride | | |
| | | | compound of | | |
| | | | Formula 1 was | | |
| | | | diluted with | | |
| | | | 5.76 mL | | |
| | | | of sterile | | |
| | | | distilled | | |
| | | | water to | | |
| | | | obtain a final | | |
| | | | volume of 6 | | |
| | | | mL. | | |
| Irinotecan | 5 | 1 | 3.18 mg | Solution | Fresh |
| | | | (irinotecan | | before |
| | | | 3 mg) was | | adminis- |
| | | | weighed | | tration |
| | | | and dissolved | | |
| | | | in 3 mL | | |
| | | | of 0.9 % | | |
| | | | saline | | |

### 3.6. Tumor Volume and Growth Curve

The average tumor growth curves at different time points are shown in FIG. 3 and Table 5.

TABLE 5

| | Drug | Tumor volume (mm³, Mean ± SEM) | | | | | | |
|---|---|---|---|---|---|---|---|---|
| Division | administered | 0 day | 3^{rd} day | 7^{th} day | 10^{th} day | 14^{th} day | 17^{th} day | 21^{st} day |
| Example 3 | Hydrochloride | 156.55 ± | 170.62 ± | 209.68 ± | 261.22 ± | 327.46 ± | 383.98 ± | 487.84 ± |
| | compound of | 5.69 | 7.95 | 15.23 | 15.05 | 22.63 | 22.49 | 33.25 |
| | Formula 1 + | | | | | | | |
| | irinotecan | | | | | | | |
| Comparative | Vehicle | 156.63 ± | 185.29 ± | 257.49 ± | 399.38 ± | 537.06 ± | 703.05 ± | 905.67 ± |
| Example 3 | | 7.36 | 9.13 | 18.73 | 32.92 | 45.47 | 53.88 | 52.20 |
| Comparative | Hydrochloride | 156.60 ± | 190.75 ± | 272.69 ± | 359.75 ± | 461.91 ± | 588.26 ± | 727.36 ± |
| Example 4 | compound of | 5.55 | 12.41 | 23.71 | 28.73 | 36.12 | 53.69 | 69.46 |
| | Formula 1 | | | | | | | |
| | (2 mg/kg) | | | | | | | |
| Comparative | Irinotecan | 156.47 ± | 169.14 ± | 211.71 ± | 292.97 ± | 404.08 ± | 495.30 ± | 643.86 ± |
| Example 5 | | 5.64 | 7.04 | 12.86 | 22.57 | 23.29 | 29.25 | 40.67 |

Referring to Table 5, Example 3 showed much less growth of tumor volume compared to Comparative Examples 4, 5. That is, it was confirmed that, in the experiment using mice, the pharmaceutical composition for co-administration of the present disclosure significantly reduced the volume growth of the tumor compared to the administration of the anticancer drug alone.

### 3.7. Inhibition of Tumor Growth

In the treatment of subcutaneous MKN45 human gastric cancer xenograft model, the inhibition of tumor growth by a single preparation of the hydrochloride compound of Formula 1 and a combination preparation of the hydrochloride compound of Formula 1 and irinotecan was measured, and results are shown in Table 6.

TABLE 6

| | | Tumor size | | Tumor | |
|---|---|---|---|---|---|
| | | on | | growth | |
| | | 21^{st} day | | inhi- | |
| | | (mm³, | | bition | |
| | Drug | Mean ± | T/C | (TGI, | |
| Division | administered | SEM) | (%) | %) | p-value |
| Example 3 | Hydrochloride | 487.84 ± 33.25 | 53.87 | 46.13 | <0.001 |
| | compound | | | | |
| | of Formula | | | | |
| | 1 + irinotecan | | | | |
| Comparative | Vehicle | 905.67 ± 52.20 | — | — | — |
| Example 3 | | | | | |
| Comparative | Hydrochloride | 727.36 ± 69.46 | 80.31 | 19.69 | 0.127 |
| Example 4 | compound | | | | |
| | of Formula | | | | |
| | 1 (2 mg/kg) | | | | |
| Comparative | Irinotecan | 643.86 ± 40.67 | 71.09 | 28.91 | 0.008 |
| Example 5 | | | | | |

Referring to Table 6, when using the combination of hydrochloride compound of Formula 1 of Example 3 (2 mg/kg, QD) and irinotecan (5 mg/kg, QW) in the MKN45 model, the tumor size was 487.84 mm³ on the 21st day and the TGI (Tumor Growth Inhibition) value was 46.13% on the 21st day. Meanwhile, Comparative Example 3 using the vehicle control group showed the average tumor size reaching 905.67 mm³ on the 21st day, Comparative Example 4 in which 2 mg/kg of the hydrochloride compound of Formula 1 was administered as QD showed the TGI value of 19.69% on the 21st day, and Comparative Example 5 in which 5 mg/kg of irinotecan was administered as QW showed the TGI value of 28.91%. Here, TGI is calculated as TGI %=(1-AT/AC)×100 (wherein T and C refer to an average tumor volume of the treatment group and the control group, respectively). In vivo TGI of 70% or more may be regarded to be sensitive to treatment, in vivo TGI between 40% and 70% may be regarded to be partially responsive to a progressive disease, and in vivo TGI of 40% or less may be regarded to be non-responsive (Cancer Res 2006; 66: (14). Jul. 15, 2006, Chronic Administration of Valproic Acid Inhibits Prostate Cancer Cell Growth In vitro and In vivo).

That is, the combination of hydrochloride compound of Formula 1 of the present disclosure and irinotecan may be regarded to exhibit significant anti-tumor efficacy compared to the vehicle control group, the single preparation of the hydrochloride compound of Formula 1, and the single preparation of irinotecan.

### Experimental Example 4

### Objectives of Experiment

The primary objective of the present experiment is to evaluate the safety, maximum tolerated dose (MTD), and recommended phase 2 dose (RP2D) of the combination of the pharmaceutical compositions of the present disclosure for patients with advanced gastric cancer.

The secondary purpose of the present experiment is: to assess the efficacy of the combination of the pharmaceutical compositions of the present disclosure as defined by objective response rate (ORR), progression-free survival (PFS), disease control rate (DCR), duration of response (DOR), time to response (TTR), best overall response (BOR), time to progression (TTP), and overall survival (OS) as evaluated by Response Evaluation Criteria in Solid Tumors (RECIST) v1.1; to evaluate pharmacokinetics (PK) of single ascending dose (SAD) of the compound of Formula 1 when given in combination with the compound of Formula 1 and irinotecan in patient with advanced gastric cancer; and to evaluate the population PK modeling of the compound of Formula 1 and irinotecan when given in combination with the compound of Formula 1 and irinotecan in patient with advanced gastric cancer.

### Endpoints of Experiment

Primary endpoint of the experiment is; to determine MTD and RP2D of the pharmaceutical compound of the present disclosure; and to determine the occurrence of DLT and the incidence of adverse event (AE)/serious adverse event (SAE).

Secondary endpoint of the experiment are related to safety and efficacy, wherein the safety endpoint refers to changes from the baseline in safety parameters including physical examination, vital signs and electrocardiogram (ECG), and clinical laboratory results including hematology, coagulation test, clinical chemistry, and urinalysis, and the efficacy endpoint refers to the number of patients with ORR, PFS, BOR, TTP, DCR, DOR, TTR, and OS as evaluated by RECIST v1.1.

Exploratory endpoint of the experiment is to assess the correlation between the efficacy of the compound of Formula 1 and ATM levels (high or low) in patients with advanced gastric cancer.

### Design of Experiment

Each part of the experiment was divided into three periods: Screening, Treatment, and Follow-up (including Survival Follow-up) to proceed.

Here, a dose finding includes a group of “compound of Formula 1+irinotecan”.

TABLE 7
Dose finding part A

| Compound of Formula 1 | | | |
|---|---|---|---|
| | Schedule | Dose/schedule | Number |
| | (1 cycle = 14 | of irinotecan | of |
| Dose | days*) | (1 cycle = 14 days*) | patients |
| 40 mg | QD, Days 1-7 ** | 50 mg/m² IV on Day 1*** | 3 to 6 |
| 40 mg | QD, Days 1-7 ** | 75 mg/m² IV on Day 1*** | 3 to 6 |
| 40 mg | QD, Days 1-7 ** | 100 mg/m² IV on Day 1*** | 3 to 6 |
| 80 mg | QD, Days 1-7 ** | | 3 to 6 |
| 120 mg | QD, Days 1-7 ** | | 3 to 6 |
| 120 mg | QD, Continuous** | | 3 to 6 |
| 160 mg | QD, Continuous** | | 3 to 6 |

Abbreviations: IV = intravenous; QD = once a day

Dose finding part B

| Compound of Formula 1 | | Dose/schedule | Dose/ | Num- |
|---|---|---|---|---|
| | Schedule | of irinotecan | schedule of | ber |
| | (1 cycle = 14 | (1 cycle = | PEGylated | of |
| Dose | days*) | 14 days*) | G-CSF | patients |
| 40 mg | QD, Days | 100 mg/m² IV | prophylact | 3 to 6 |
| | 1-7 ** | on Day 1 | ically at | |
| 20 mg | QD, Days | | least 24 | 3 to 6 |
| | 1-7 ** | | hours post | |
| 10 mg | QD, Days | | irinotecan | 3 to 6 |
| | 1-7 ** | | infusion | |

Reference:
As starting doses, 120 mg of Compound of Formula 1 + irinotecan (part A).
*Administration cycle may be 14 days or 21 days in one cycle.
**: The number of days of administration of the compound of Formula 1 may be 1 to 7 or 1 to 5 or 2 to 6.
***: The number of days of administration of the irinotecan may be 1 or 2 to 6.

For dose expansion, about 45 patients with advanced gastric cancer (15 patients per group) who met all eligibility criteria were enrolled in the open dose expansion part to further explore the safety, tolerability, and efficacy of the compound of Formula 1 in combination with irinotecan.

Patients were administered with an appropriate dose (potential RP2D) of the compound of Formula 1 as QD in combination with irinotecan in 14-day cycle until the disease progression, unacceptable toxicity, or another withdrawal criterion is met. Treatment cycles will occur consecutively without interruption except when necessary to manage AEs. When an investigator determines that it is clinically beneficial to the patients to continue the treatment even after disease progression has been confirmed, the treatment using the compound of Formula 1 can be continued.

### Selection of Experimental Population (Selection of Patients)

The experimental population of the present disclosure includes patients with advanced gastric cancer, as defined by metastatic or recurrent/refractory advanced disease in third-line or fourth line treatment. The patients in the dose finding part must have evaluable disease. The patients in the dose expansion part must have measurable disease at baseline by imaging methods (computed tomography [CT] or magnetic resonance imaging [MRI]).

The criteria for patient selection are as follows.
Patients who have signed the informed consent form (ICF) for screening approved by the Independent Ethics Committee (IEC)/Institutional Review Board (IRB) before performing any clinical trial procedures.
Males or females at least 18 years at the time of consent (i.e., screening), or people subject to local regulatory requirements when the legal age to provide consent to participate in the trial is over 18.
Patients with recurrent or advanced metastatic gastric cancer including gastroesophageal junction or upper part of the stomach, who were treated ≥2 lines with palliative chemotherapy before screening.
At least 1 evaluable lesion for the dose finding part and at least 1 measurable lesion according to RECIST v1.1 for the dose expansion part.
Eastern Cooperative Oncology Group (ECOG) performance status of ≤1.
Life expectancy of at least 12 weeks at the time of enrollment.
Capable of oral administration.
Patients with adequate organ function including whose clinical laboratory assessment results conducted within the last 1 week prior to study drug administration.
A negative serum pregnancy test result within 4 weeks prior to the study drug administration is required for pre-menopausal women. Also, patients should have a negative urine pregnancy test result before study drug administration on Cycle 1 Day 1. Women with no childbearing potential because of surgical sterility or ≥1 year having passed after menopause can be included without a serum and/or urine pregnancy test.
Men and women with childbearing potential must agree to use adequate contraception starting with the first dose of study therapy through 6 months after the last dose of study therapy as instructed by the investigator. Effective contraception is defined as a method resulting in a low failure rate (i.e., <1% per year) when used consistently and accurately (example: implant, injection, combined oral contraceptive, or intrauterine device). As chosen by an investigator, acceptable contraception can include complete abstinence if a patient's lifestyle is in line with it (periodic abstinence [example: calendar, ovulation, symptomatic, post ovulation method] and withdrawal are not acceptable contraception).
Willing and able to comply with all the requirements of the protocol, including prohibitions and restrictions specified in the study protocol.

### Administration

The compound of Formula 1 was provided in 5 mg, 10 mg, 20 mg, and 40 mg tablets. The compound of Formula 1 and irinotecan were provided as a sterile, pale yellow, clear, aqueous solution at a concentration of 20 mg/ml, intended for dilution with 5% dextrose injection, United States Pharmacopoeia (USP), or a 0.9% sodium chloride injection USP, before IV infusion. The preferred diluent is 5% dextrose injection, USP.

The administration cycle and administration dose of the compound of Formula 1 and irinotecan are as shown in Table 7.

The compound of Formula 1 was administered as QD with at least 8 ounces (oz) (about 240 mL) of water. Irinotecan was administered through IV injection over 90 minutes after completion of all study procedures.

### Assessments of Efficacy

The efficacy assessments included ORR, PFS, DCR, DOR, TTR, BOR, TTP, and OS evaluated per RECIST v1.1.

Radiographic tumor assessment included all known or suspected disease sites per RECIST v1.1.

The survival follow-up was performed for survival in all patients at least every 12 weeks after discontinuation of the study treatment. The survival monitoring continues until patient death or end of the study, whichever occurs first.

### Efficacy Analysis

ORR is defined as the proportion of patients confirmed with a partial response or complete response defined per RECIST v1.1. The analysis of ORR was performed in patients with measurable lesions at baseline. Patients with no adequate postbaseline tumor assessment at the time of the analysis of ORR are counted as non-responders. ORR ratios are provided for each treatment group, including the corresponding 95% Wilson score CI.

PFS is defined as the time from initiation of a study treatment to the date radiologically confirmed by determination of an investigator (per RECIST v1.1) for disease progression or to death from any cause, whichever comes first.

DCR is defined as the proportion of patients achieving a BOR with a confirmed complete response (CR), confirmed PR or SD (minimum period of SD is 6 weeks).

DOR is defined as the period from the date the objective response (confirmation of CR or PR) is first documented to the first documented date based on objective disease progression or death due to any cause, whichever comes first. Censoring rules are similar to PFS.

TTR is defined as the period from the start date of a study treatment to the date the objective response (confirmation of CR or PR) is first documented. Censoring rules are similar to PFS.

The confirmed BOR is defined as the best response evaluated during the period from the first administration of a test drug to progression/recurrence of disease or death. For PR or CR responders in the final BOR assessment after satisfying the first response criteria at the tumor assessment, the corresponding patients should be evaluated as PR or CR responders at the next consecutive assessment. The patients with measurable lesions at baseline and with available tumor assessment results are included in the BOR analysis, and patients with no tumor assessment after baseline are considered as non-responders.

TTP is defined as the period from the first administration of an investigational drug to first disease progression. Patients with no disease progression at the study completion (including patients who died before disease progression) or patients who are out of follow-up due to dropout are censored at the last tumor assessment date.

OS is defined as the period from the first administration of a study drug to death from any cause. Patients who are alive are censored on the date the patients are last known to be alive.

### Experimental Example 5

### Experiment Results

The BOR assessment results for the patients are shown in Table 8.

TABLE 8

| | | | | | | | | | Tx | BOR |
|---|---|---|---|---|---|---|---|---|---|---|
| Division | C2D1 | C3D1 | C4D1 | C5D1 | C6D1 | C8D1 | C9D1 | C18D1 | (days) | (%) |
| Patient 1 | | SD | | | SD | | SD | | 172 | SD |
| | | (0) | | | (2.3) | | (4.7) | | | (0) |
| Patient 2 | | PR | | | PR | | | | 253 | PR |
| | | (−55.6) | | | (−50.0) | | | | | (−55.6) |
| Patient 3 | | SD | | | | | | | 152 | SD |
| | | (−17.1) | | | | | | | | (−17.1) |
| Patient 4 | | SD | | | | | SD | SD | 461 | SD |
| | | (0) | | | | | (−3.6) | (−7.14) | | (−7.14) |
| Patient 5 | SD | PR | | | | PR | | | 421 | PR |
| | (−27.8) | (−38.9) | | | | (−72.2) | | | | (−72.2) |
| Patient 6 | | | SD | | CR | | | | 168 | CR |
| | | | (−13.3) | | (−46.7) | | | | | (−46.7) |

Best Overall Response (BOR): Best overall response (smallest measure) recorded from the start of the administration until the progression/relapse of disease occurs

Complete response (CR): Complete disappearance of all tumors that have been observed, and reduction of all pathological lymph nodes to less than 10 mm

Partial response (PR): At least 30% decrease in the sum of diameters

Stable disease (SD): Neither for CR, PR, and PD, i.e., the state as it is

Progressive disease (PD): At least 20% increase in the sum of diameters, absolute increase of at least 5 mm in the sum of diameters, and appearance of one or more new lesions

According to the systemic review, stage 3 gastric cancer is known to show unsatisfactory results based on objective response rate (ORR=CR+PR) of 3% to 6% and PFS of only 1.61 months to 3.67 months in the standard of care. However, according to the experimental results above, it was confirmed that the combination of the pharmaceutical compositions of the present disclosure had an objective response rate of 33.33%, reaching 3 out of 9 patients, and median duration of treatment exceeding 5 months, thereby (5.6 months as of end of 2022) exhibiting remarkably excellent results.

1. A combination of pharmaceutical compositions for co-administration to prevent or treat cancer, wherein the combination comprises: (i) a first pharmaceutical composition comprising, as an active ingredient, a compound represented by Formula 1 or a pharmaceutically acceptable salt thereof; 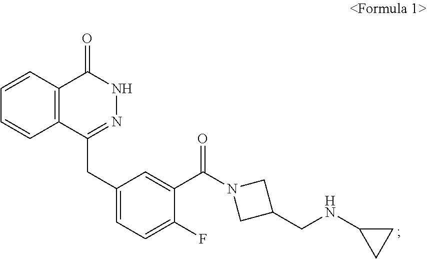 [SMILES: O=C(c1cc(Cc2n[nH]c(=O)c3ccccc23)ccc1F)N1CC(CNC2CC2)C1]
and
(ii) a second pharmaceutical composition comprising, as an active ingredient, at least one anticancer drug.

2. The combination of claim 1, wherein the anticancer drug is selected from the group consisting of a chemotherapy drug, a targeted therapy drug, a immunotherapy drug, and a hormone therapy drug.

3. The combination of claim 2, wherein the chemotherapy drug is selected from the group consisting of an alkylating agent, an antimetabolite, an anti-tumor antibiotic, a Topoisomerase inhibitor and a Mitotic inhibitor.

4. The combination of claim 2, wherein the targeted therapy drug is selected from the group consisting of an angiogenesis inhibitor, a monoclonal antibody, a proteasome inhibitor and a signal transduction inhibitor.

5. The combination of claim 2, wherein the immunotherapy drug is selected from the group consisting of a checkpoint inhibitor, a chimeric antigen receptor (CAR) T-cell therapy and a cytokine.

6. The combination of claim 2, wherein the hormone therapy drug is selected from the group consisting of an aromatase inhibitor, a selective estrogen receptor modulators, an Estrogen receptor antagonist, a luteinizing hormone-releasing hormone agonist, an anti-androgen and a CYP17 inhibitor.

7. The combination of claim 1, wherein the anticancer drug is selected from irinotecan and oxaliplatin.

8. The combination of claim 1, wherein the anticancer drug is irinotecan.

9. The combination of claim 1, wherein the pharmaceutically acceptable salt is hydrochloride.

10. The combination of claim 1, wherein the pharmaceutical compositions for co-administration are administered in which the first pharmaceutical composition and the second pharmaceutical composition are in a form of a mixture.

11. The combination of claim 1, wherein the pharmaceutical compositions for co-administration are administered in which the first pharmaceutical composition and the second pharmaceutical composition are each formulated and administered simultaneously or sequentially.

12. The combination of claim 1, wherein the first pharmaceutical composition and the second pharmaceutical composition are each comprised in the form of a separate dosage form.

13. The combination of claim 1, wherein the first pharmaceutical composition is in an oral dosage form, and the second pharmaceutical composition is in an injection dosage form.

14. The combination of claim 1, wherein the first pharmaceutical composition is to be administered once daily.

15. The combination claim 1, wherein the first pharmaceutical composition comprises about 5 mg, about 10 mg, about 20 mg, about 30 mg, about 40 mg, or about 50 mg of the compound represented by Formula 1.

16. The combination of claim 1, wherein the first pharmaceutical composition is to be dosed at about 1 mg to about 180 mg per administration.

17. The combination of claim 1, wherein the first pharmaceutical composition is to be dosed at about 10 mg, about 20 mg, about 40 mg, about 80 mg, about 120 mg, or about 160 mg per administration.

18. The combination of claim 1, wherein the second pharmaceutical composition is to be intravenously dosed at about 30 mg/m² to about 120 mg/m².

19. The combination of claim 1, wherein the second pharmaceutical composition is to be intravenously dosed at about 50 mg/m², about 75 mg/m², or about 100 mg/m².

20. The combination of claim 1, wherein the first pharmaceutical composition is to be dosed as a cycle, wherein the cycle comprises administering the first pharmaceutical composition for about 14 days or about 21 days.

21. The combination of claim 1, wherein the second pharmaceutical composition is to be dosed as a cycle, wherein the cycle comprises administering the second pharmaceutical composition for about 14 days or about 21 days.

22. The combination of claim 1, wherein the second pharmaceutical composition is to be dosed as a cycle, wherein the cycle is repeated at least once.

23. The combination of claim 1, wherein the cancer is gastric cancer.

24. A method of preventing or treating cancer, the method comprising co-administering the combination of claim 1 to a subject in need thereof.

25. The method of claim 24, wherein the cancer is gastric cancer.

26. A kit for prevention or treatment of cancer, comprising the combination of claim 1, wherein the first pharmaceutical composition is to be administered simultaneously, separately, or sequentially.

27. The kit of claim 26, wherein the first pharmaceutical composition is present in a single dosage form and the second pharmaceutical composition is present in a separate dosage form.
